(19) United States
(12) Patent Application Publication
(10) Pub. No.: US 2009/0233324 A1
(43) Pub. Date: Sep. 17, 2009

(54) Methods for Diagnosing Cancer Using Samples Collected From A Central Vein Location or an Arterial Location

(76) Inventor: Anne R. Kopf-Sill, Portola Valley, CA (US)

Correspondence Address: WILSON SONSINI GOODRICH & ROSATI, 650 PAGE MILL ROAD, PALO ALTO, CA, 94304-1050, US

(21) Appl. No.: 12/046,427

(22) Filed: Mar. 11, 2008

(51) Int. Cl. C12Q 1/02; C12N 5/06

(52) U.S. Cl. 435/29; 435/325

(57) ABSTRACT

The invention encompasses methods for selectively enriching in rare particles from blood samples harvested from the vein jugular vein the femoral vein, the subclavian vein, or an artery. These rare particles can be circulating tumor cells, circulating stem cells, or fragments thereof. Blood samples harvested from different sources can contain higher or lower concentrations of rare particles. The rare particles can be enriched by applying the blood samples to a microfluidic device with a two dimensional array of obstacles.

### TECHNICAL FIELD

The invention is related to medical diagnostics and methods of sampling blood for enrichment of rare particles.

### BACKGROUND

Cancer is a disease marked by the uncontrolled proliferation of abnormal cells. In normal tissue, cells divide and organize within the tissue in response to signals from surrounding cells. Cancer cells do not respond in the same way to these signals, causing them to proliferate and, in many organs, form a tumor. As the growth of a tumor continues, genetic alterations may accumulate, manifesting as a more aggressive growth phenotype of the cancer cells. If left untreated, metastasis, the spread of cancer cells to distant areas of the body by way of the lymph system or bloodstream, may ensue. Metastasis results in the formation of secondary tumors at multiple sites, damaging healthy tissue. Most cancer death is caused by such secondary tumors.

Despite decades of advances in cancer diagnosis and therapy, many cancers continue to go undetected until late in their development. As one example, most early-stage lung cancers are asymptomatic and are not detected in time for curative treatment, resulting in an overall five-year survival rate for patients with lung cancer of less than 15%. However, in those instances in which lung cancer is detected and treated at an early stage, the prognosis is much more favorable.

Therefore, there exists a need to develop new methods for detecting cancer at earlier stages in the development of the disease.

### INCORPORATION BY REFERENCE

All publications and patent applications mentioned in this specification are herein incorporated by reference to the same extent as if each individual publication or patent application was specifically and individually indicated to be incorporated by reference.

### SUMMARY OF THE INVENTION

The invention provides for methods to selectively concentrate one or more rare particles from an organism comprising: applying a blood sample taken from a jugular vein, a femoral vein, a subclavian vein, an artery or a heart to a device that selectively enriches the one or more rare particles. The artery can be a radial artery, an ulnar artery, a brachial artery, a femoral artery, a carotid artery, a subclavian artery or a brachiocephalic artery. The method can be used for blood samples taken from human or non-human animals. The rare particles can be a variety of rare particles, including circulating tumor cells, circulating stem cells or fragments thereof.

The device used for enrichment of the one or more rare particles can be a flow-through device. The flow-through device can selectively capture the one or more rare particles or allow for the one or more rare particles to be directed away from other particles. In some embodiments of the invention, the device used to selectively enrich the one or more rare particles is a microfluidic device. The microfluidic device can include an array of obstacles. One variation can include functionalizing the array of obstacles with binding moieties. These binding moieties specifically bind EPCAM, E-Cadherin, Mucin-1, Cytokeratin, EGFR, LAR, CD34, or folate receptor.

Another aspect of the invention provides for methods for diagnosing, prognosing, or theranosing cancer in a subject comprising a) enriching one or more circulating tumor cells or fragments thereof from a blood sample taken from a jugular vein, a femoral vein, a subclavian vein, an artery or a heart and b) diagnosing, prognosing, or theranosing based on analysis of the enriched one or more circulating tumor cells or fragments thereof. The artery can be a radial artery, an ulnar artery, a brachial artery, a femoral artery, a carotid artery, a subclavian artery or a brachiocephalic artery. The enriching step can comprise applying the blood sample to a microfluidic device that selectively captures the one or more circulating tumor cells or fragments thereof. In some embodiments of the invention, the selective capture of the one or more circulating tumor cells is based on affinity, size, shape or deformability. The microfluidic device can include a two-dimensional array of obstacles and/or binding moieties for selectively binding the one or more circulating tumor cells or fragments thereof. The method of diagnosing, prognosing, or theranosing cancer can further comprise enumerating the one or more circulating tumor cells or fragments thereof.

Other goals and advantages of the invention will be further appreciated and understood when considered in conjunction with the following description and accompanying drawings. While the following description may contain specific details describing particular embodiments of the invention, this should not be construed as limitations to the scope of the invention, but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### BRIEF DESCRIPTION OF THE FIGURES

FIG. 1 is an illustration showing the blood sampling locations and the protocol for analysis of the blood samples.

FIG. 2 is a diagram showing harvested mouse bodies and the corresponding results from analysis of blood samples taken on day 9.

FIG. 3 is a diagram showing harvested mouse bodies and the corresponding results from analysis of blood samples taken on day 16.

FIG. 4 is a compilation of photomicrographs of cardiac puncture blood samples visualized on a glass slide.

FIG. 5 is a depiction of the results obtained by scanning circulating tumor cells from a cardiac puncture blood sample that were enriched using a microfluidic device with a two-dimensional array of obstacles.

### DETAILED DESCRIPTION OF THE INVENTION

Overview of the Invention

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The invention features methods for selectively concentrating one or more rare particles in a blood sample by applying a blood sample taken from a large central vein or an artery to a device for selective enrichment of the one or more rare particles. The large central vein can be a jugular vein, a femoral vein or a subclavian vein. The one or more rare particles can be one or more circulating tumor cells, circulating stem cells or fragments thereof.

In another embodiment of the invention, the invention features methods for diagnosing, prognosing, or theranosing cancer in a subject comprising the steps of a) enriching in one or more circulating tumor cells or fragments thereof from a blood sample taken from a jugular vein, a femoral vein, a subclavian vein, or an artery and b) diagnosing, prognosing, or theranosing based on analysis of the enriched one or more circulating tumor cells or fragments thereof.

Cellular Samples

Cellular samples collected from a subject for diagnosis of cancer state can be collected from a variety of sources. The sources of blood can be obtained from humans or non-human animals. Blood sampled from different locations can have a higher or lower concentration of a rare particle due to exposure of the blood to different environments. The concentration of rare particle in the blood can change as fluids from the lymphatic system are drained into the blood, as blood is exposed to cellular tissue containing rare particles or as blood passes through capillary beds. The location of the puncture for blood sampling can be the same as the location where the blood is collected. Alternatively, the location of the puncture for blood sampling can be different from the location where the blood is collected. For example, a femoral artery can be punctured and blood can be collected from the heart.

The present invention is directed toward the use of blood samples collected from a central vein location or an artery. A blood sample from a central vein location can include, for example, blood taken from a central venous port, a central venous catheter, a large vein of the neck, chest, or groin including, but not limited to, a jugular vein, a subclavian vein, and a femoral vein. A blood sample from an artery can include, for example, blood taken from a heart, an arm or a leg. The artery can be any artery, for example an arterial location can be a radial artery, an ulnar artery, a brachial artery, or a femoral artery. The blood can also be sampled from a cardiac space.

Circulating Tumor Cells

Epithelial cells that are exfoliated from solid tumors have been found in very low concentrations in the circulation of patients with advanced cancers of the breast, colon, liver, ovary, prostate, and lung, and the presence or relative number of these cells in blood has been correlated with overall prognosis and response to therapy. These exfoliated epithelial cells, also called circulating tumor cells, may be an early indicator of tumor expansion or metastasis before the appearance of clinical symptoms.

Circulating tumor cells typically have a short half-life of approximately one day, and their presence generally indicates a recent influx from a proliferating tumor. Therefore, circulating tumor cells represent a dynamic process that may reflect the current clinical status of patient disease and therapeutic response. In some cases, circulating tumor cells can lyse and/or undergo apoptosis, leaving fragments of circulating tumor cells. Enumeration and characterization of circulating tumor cells and fragments thereof, using the methods of the invention, is useful in assessing cancer diagnosis, prognosis and/or theranosis. Theranosis can include monitoring therapeutic efficacy for early detection of treatment failure that may lead to disease relapse. In addition, circulating tumor cell analysis according to the invention enables the detection of early relapse in presymptomatic patients who have completed a course of therapy.

Circulating Stem Cells

Stem cells can circulate in the blood for repair of damaged cellular tissue. These circulating stem cells have numerous applications. For example, stem cells can be collected from cancer patients prior to delivery of chemotherapy for redelivery to the patient after chemotherapy. In other embodiments of the invention, circulating stem cells and fragments thereof can be harvested for analytical purposes. The collection of circulating stem cells and fragments thereof can be performed by harvesting stem cells from bone marrow or blood taken from the patient. Collection of stem cells from blood offers several clinical advantages, but is impeded by the low concentration of circulating stems cells found in blood samples. To address this issue, selective enrichment of circulating stem cells and fragments thereof can be performed using the methods of the present invention.

Enrichment Methods

Rare particles, such as circulating tumor cells, circulating stem cells and/or fragments thereof can be enriched from a pool of blood cells separation based on size, affinity, shape or deformability. Circulating tumor cells are generally larger than most blood cells. This allows for selective enrichment of circulating tumor cells based on size. Rare particles such as circulating tumor cells, circulating stem cells and fragments thereof present specific cell surface antigens. Binding moieties to these cell surface antigens can be used for selective enrichment. For example, circulating tumor cells can present a high abundance of epithelial cell adhesion molecule (EpCAM) and circulating stem cells can present a high abundance of CD34 surface antigen. Previous methods and devices using selective enrichment of rare particles based on size, affinity, shape or deformability have been described and are hereby incorporated by reference. These references include Nagrath et al. Nature 450, 1235-1241 (2007), co-pending U.S. application Ser. No. 11/323,962, US Application No. 2006/0252054.

In some embodiments of the invention, one or more rare particles can be enriched using a flow-through device. The blood sample can be applied to a flow-through device, wherein the one or more rare particle is captured in the flow-through device. In alternate embodiments of the invention, the blood sample can be applied to a flow-through device, wherein the one or more rare particle is directed away from other particles in a continuous mode.

The device for enriching the rare particles can be a microfluidic device. A variety of features in the microfluidic device can be used for enrichment of the one or more rare particles, including microchannels, microfiltration, micro-obstacles, or affinity interaction. In some embodiments of the invention, the microfluidic device includes a two-dimensional array of obstacles. The obstacles can have a random or non-random arrangement. The obstacles can be arranged to have pinch points, where the gap between obstacles is less than the gap between other nearby obstacles. These pinch points can allow for selective capture of rare particles. In other embodiments of the invention, the obstacles can be functionalized with one or more binding moieties. These binding moieties can specifically bind EPCAM, E-Cadherin, Mucin-1, cytokeratin, EGFR, LAR, CD34 or folate receptor.

Analysis Methods

The methods for analysis of the enriched one or more rare particles can comprise enumerating the enriched one or more rare particles. The enumeration can include indicating the enriched one or more rare particles using a dye, wherein the dye can be fluorescent or non-fluorescent. The enriched one or more rare particles can be detected using a fluorometer, a luminometer, or spectrometer. In other embodiments of the invention, the enriched one or more rare particles can be imaged using any imaging device. An imaging device, for example, can be a microscope. For example, the microscope can be a fluorescent microscope.

### EXAMPLE 1

A mouse study evaluating three different bleed sites were for recovery of circulating tumor cells (CTC) was performed.

Sample Collection

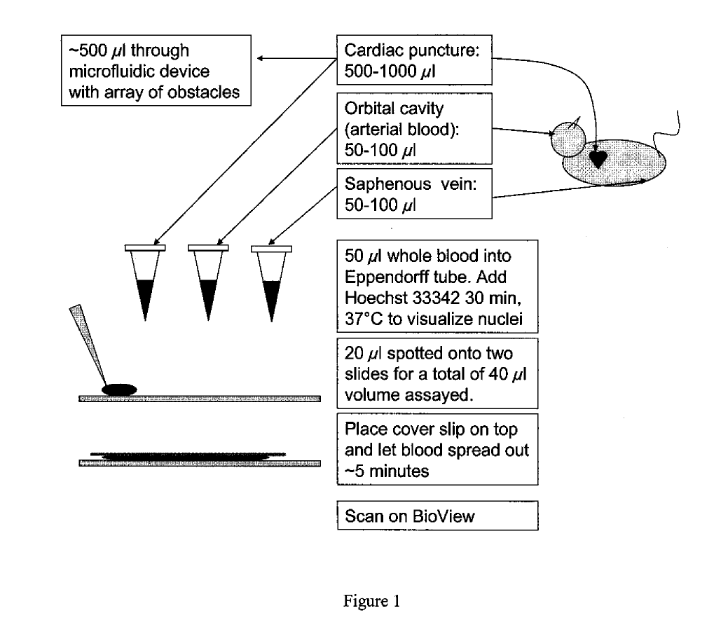

At two time points, three groups of mice, including two positive control mice and one negative control mice, were sampled. Positive control mice were subjected to injection of GFP producing tumors. Blood samples were obtained via a saphenous vein, a retro-orbital bleed (arterial blood) and cardiac puncture. An illustration depicting the locations for blood sampling is shown in FIG. 1. One group of mice was terminated and primary tumor and metastatic tissue was collected.

Analysis

Each blood sample was evaluated for amount of circulating tumor cells prior to enrichment using a photomicroscope. For the cardiac puncture samples, circulating tumor cells were selectively enriched from the blood sample. The enrichment was performed using a microfluidic device with a two-dimensional array of obstacles that contained pinch points and were functionalized with an EPCAM binding moiety. The circulating tumor cells were then enumerated by analysis of images obtained using a photomicroscope.

### Protocol for Analysis of Non-Enriched Blood Samples

The protocol for analysis of non-enriched blood samples is illustrated in FIG. 1. Cells were stained by transferring 50 μL of blood sample to an Eppendorf tube. Hoescht 33342 dye was added to the blood sample for 30 minutes and incubated at 37° C. to visualize the nuclei. 20 μL of the stained blood sample was spotted on two glass slides. A cover slip was placed on the blood sample, and the blood sample was allowed to spread for about 5 minutes. The glass slide was then scanned on a BioView instrument.

Results

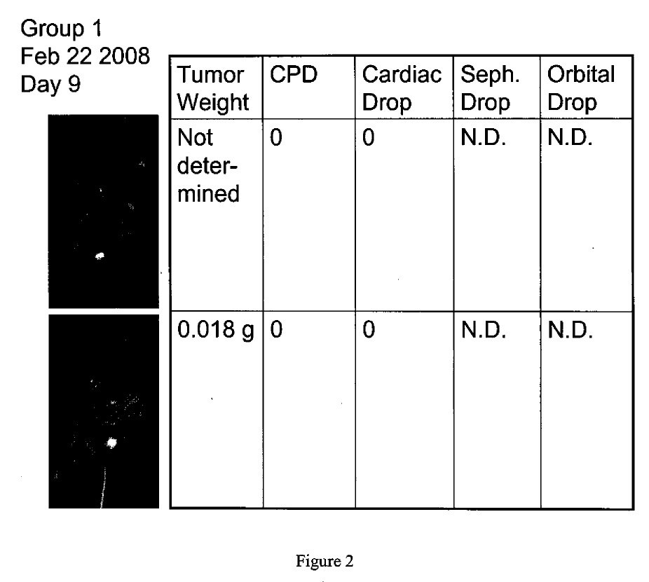

Samples were harvested from mice on day 9. Data collected from samples taken on day 9 are shown in FIG. 2. Day 9 results showed that while GFP producing tumors were visualized using fluorescent light, no circulating tumor cells were collected. The total amount of tumor weight obtained in one mice was 0.018 grams.

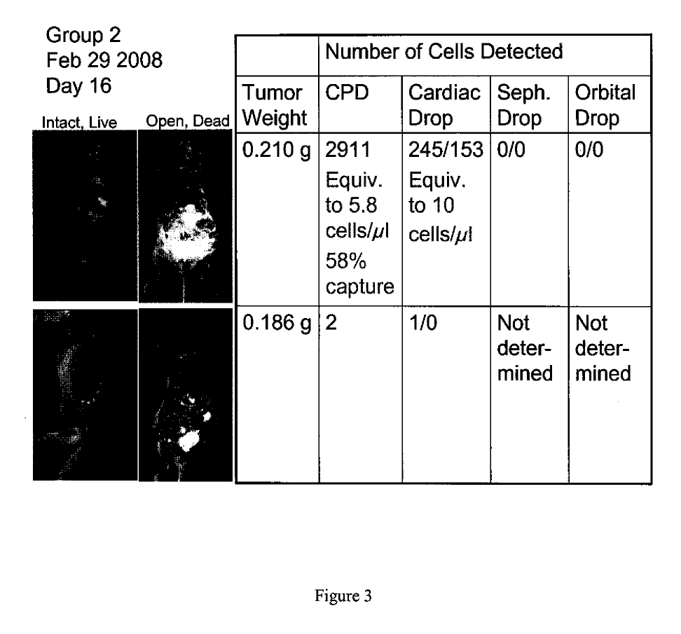

Samples were harvested from mice on day 16. Data collected from samples taken on day 16 are shown in FIG. 3. Analysis of live mice using fluorescent light showed significant accumulation of GFP producing tumor. Analysis of open and dead mice showed that even greater amounts of GFP producing tumors could be visualized using fluorescent light. In one mouse, approximately 0.2 grams of GFP producing tumor was harvested. For the same mouse, the number of circulating tumor cells detected in the cardiac puncture blood samples prior to enrichment was approximately 10 cells per μL of initial blood sample. This blood sample allowed for approximately 200 circulating tumor cells to be visualized in a 20 μL sample. In comparison, approximately 2900 circulating tumor cells were visualized after enrichment of the cardiac puncture sample from the same mouse using the microfluidic device described above. This corresponded to approximately 5.8 circulating cells per μL of initial blood sample or 58% cell capture.

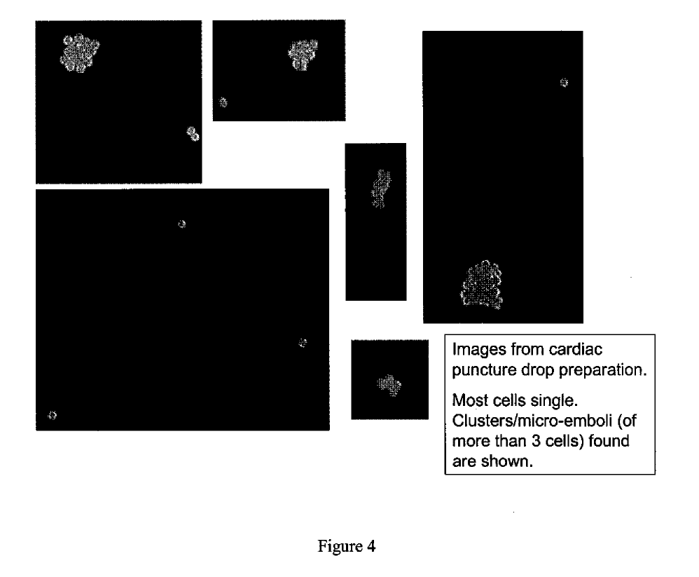

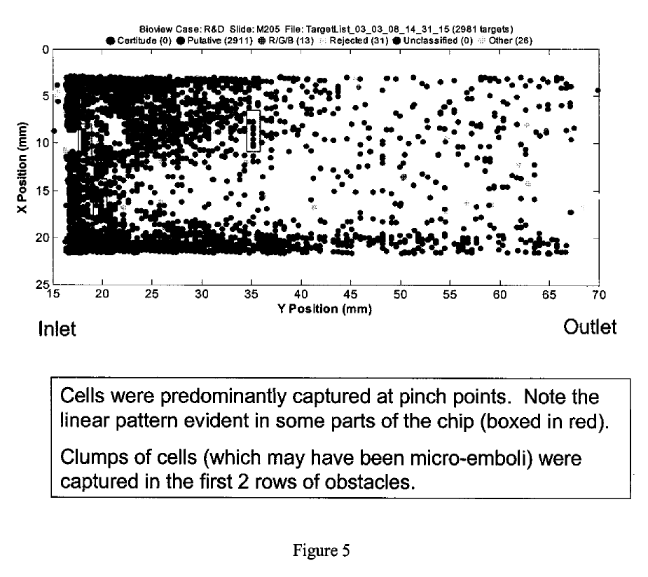

As shown in FIG. 4, images of the blood samples taken by cardiac puncture show that some cells exist as clusters or micro-emboli of 3 or more cells. These clusters or micro-emboli were also captured during enrichment using the microfluidic device described above. As shown in FIG. 5, many of these clumps or clusters of cells were captured in the first two rows of obstacles.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method for selectively concentrating one or more rare particles from an organism comprising: applying a blood sample taken from a jugular vein, a femoral vein, a subclavian vein, an artery or a heart to a device that selectively enriches the one or more rare particles.

2. The method of claim 1, wherein the artery is a radial artery, an ulnar artery, a brachial artery, a femoral artery, a carotid artery, a subclavian artery or a brachiocephalic artery.

3. The method of claim 1, wherein the organism is a human.

4. The method of claim 1, wherein the one or more rare particles comprise one or more circulating tumor cells, circulating stem cells, or fragments thereof.

5. The method of claim 1, wherein device is a flow-through device.

6. The method of claim 3, wherein said device selectively retains said rare particles.

7. The method of claim 1, wherein said device is a microfluidic device.

8. The method of claim 7, wherein the microfluidic device comprises a two-dimensional array of obstacles.

9. The method of claim 7, wherein the microfluidic device is functionalized with binding moieties.

10. The method of claim 9, wherein the binding moieties specifically binds EPCAM, E-Cadherin, Mucin-1, Cytokeratin, EGFR, LAR, CD34 or folate receptor.

11. A method for diagnosing, prognosing, or theranosis of cancer in a subject, said method comprising the steps of:
a) enriching one or more circulating tumor cells or fragments thereof from a blood sample taken from a jugular vein, a femoral vein, a subclavian vein, an artery or a heart and
b) diagnosing, prognosing, or theranosing based on analysis of said enriched one or more circulating tumor cells or fragments thereof.

12. The method of claim 11, wherein the artery is a radial artery, an ulnar artery, a brachial artery, a femoral artery, a carotid artery, a subclavian artery or a brachiocephalic artery.

13. The method of claim 11, wherein enriching step comprises applying the blood sample to a microfluidic device that selectively captures the one or more circulating tumor cells or fragments thereof.

14. The method of claim 13, wherein the selective capture occurs based on affinity, size, shape or deformability.

15. The method of claim 13, wherein the microfluidic device is covered by at least one binding moiety that selectively binds the one or more circulating tumor cells or fragments thereof.

16. A method of claim 11, further comprising enumerating the one or more circulating tumor cells or fragments thereof.
